(19) United States
(12) Patent Application Publication
(10) Pub. No.: US 2025/0092164 A1
(43) Pub. Date: Mar. 20, 2025

(54) MODIFIED GUAYULE RESIN PRODUCT AND RELATED PROCESSES

(71) Applicants: Mark N. DEDECKER, North Canton, OH (US); Walter A. SALAMANT, Akron, OH (US); Bridgestone Corporation, Chuo-ku (JP)

(72) Inventors: Mark N. DeDecker, North Canton, OH (US); Walter Anthony Salamant, Akron, OH (US)

(73) Assignee: Bridgestone Corporation, Chuo-ku, TK (JP)

(21) Appl. No.: 18/550,840

(22) PCT Filed: Feb. 8, 2022
(86) PCT No.: PCT/US2022/070562

§ 371 Date: Sep. 15, 2023

Related U.S. Application Data
(60) Provisional application No. 63/146,948, filed on Feb. 8, 2021.

(51) Int. Cl. C08C 19/22

(52) U.S. Cl. CPC C08C 19/22

(57) ABSTRACT

Disclosed herein are a modified guayule resin product and related processes for preparing the modified guayule resin product. The modified guayule resin product comprises a mixture of argentatins having at least one functional group, wherein the at least one functional group is provided by an isocyanate-containing functionalizing compound. In a second embodiment, a process for preparing a modified guayule resin product is provided whereby a guayule resin component (comprising a mixture of argentatins) is mixed with an isocyanate-containing functionalizing compound to produce a modified guayule resin comprising functionalized argentatins having at least one functional group provided by the isocyanate-containing functionalizing compound.

### FIELD

The present application is directed to a modified guayule resin product and related processes for preparing the modified guayule resin product.

### BACKGROUND

The guayule plant (Parthenium argentatum) is a woody shrub-like plant that produces rubber and resin. Processes which are directed to isolating rubber from the guayule plant will produce a considerable amount of guayule resin as a by-product.

### SUMMARY

Disclosed herein are a modified guayule resin product and related processes for preparing the modified guayule resin product.

In a first embodiment, a modified guayule resin product is provided. The modified guayule resin product comprises a mixture of argentatins having at least one functional group, wherein the at least one functional group is provided by an isocyanate-containing functionalizing compound.

In a second embodiment, a process for preparing a modified guayule resin product is provided. According to the process of the second embodiment, a guayule resin component is provided which comprises a mixture of argentatins having —OH groups. The guayule resin component is mixed with an isocyanate-containing functionalizing compound to produce a modified guayule resin comprising functionalized argentatins having at least one functional group provided by the isocyanate-containing functionalizing compound.

### DETAILED DESCRIPTION

Disclosed herein are a modified guayule resin product and related processes for preparing the modified guayule resin product.

In a first embodiment, a modified guayule resin product is provided. The modified guayule resin product comprises a mixture of argentatins having at least one functional group, wherein the at least one functional group is provided by an isocyanate-containing functionalizing compound.

In a second embodiment, a process for preparing a modified guayule resin product is provided. According to the process of the second embodiment, a guayule resin component is provided which comprises a mixture of argentatins having —OH groups. The guayule resin component is mixed with an isocyanate-containing functionalizing compound to produce a modified guayule resin comprising functionalized argentatins having at least one functional group provided by the isocyanate-containing functionalizing compound.

### Definitions

The terminology as set forth herein is for description of the embodiments only and should not be construed as limiting the invention as a whole.

As used herein, the term “—OH group” is intended to include —OH groups that are bonded to a carbon atom as an alcohol group (e.g., as in an argentatin molecule) but to exclude —OH groups that are bonded to a carbon atom as part of carboxyl group (i.e., —COOH).

As used herein, the term “majority” refers to more than 50% (e.g., at least 50.1%, at least 50.5%, at least 51%, etc.).

As used herein, the term “minority” refers to less than 50% (e.g., no more than 49.5%, no more than 49%, etc.).

As used herein, the abbreviation Mn is used for number average molecular weight.

As used herein, the abbreviation Mp is used for peak molecular weight.

As used herein, the abbreviation Mw is used for weight average molecular weight.

As used herein, the term “natural rubber” means naturally occurring rubber such as can be harvested from sources such as Hevea rubber trees and non-Hevea sources (e.g., guayule plant and dandelions such as TKS). In other words, the term “natural rubber” should be construed so as to exclude synthetic polyisoprene.

As used herein, the term “guayule rubber” is a sub-category of natural rubber which has been harvested from the guayule plant. In contrast, natural rubber which has not been harvested from the guayule plant is referred to herein as “non-guayule natural rubber” and can include Hevea rubber as well as other sources such as dandelion.

As used herein the term “polyisoprene” means synthetic polyisoprene. In other words, the term is used to indicate a polymer that is manufactured from isoprene monomers, and should not be construed as including naturally occurring rubber (e.g., Hevea natural rubber, guayule-sourced natural rubber, or dandelion-sourced natural rubber). However, the term polyisoprene should be construed as including polyisoprenes manufactured from natural sources of isoprene monomer.

### Modified Guayule Resin Product

As discussed above, the first embodiment disclosed herein is directed to a modified guayule resin product and the second embodiment disclosed herein provides a process for preparing a modified guayule resin product. According to the first and second embodiments, the modified guayule resin product comprises a mixture of argentatins having at least one functional group, wherein the at least one functional group is provided by an isocyanate-containing functionalizing compound. This mixture of argentatins having at least one functional group, wherein the at least one functional group is provided by an isocyanate-containing functionalizing compound is also referred to herein as a “mixture of functionalized argentatins” or simply as “functionalized argentatins.” As those of skill in the art will understand, argentatins constitute a class of compounds which naturally occur in guayule resin and which contain (in unmodified or non-functionalized form) one or more —OH groups. These argentatins have been categorized into various categories including Argentatin A, Argentatin B, Argentatin C, Argentatin D, Argentatin E, and Argentatin F. The modification or functionalization of the argentatins takes place through bonding of the oxygen atom from an —OH group of an argentatin compound to the carbon atom in the carbonyl of the isocyanate group of the isocyanate-containing functionalizing compound. In other words, the point of attachment between the argentatin compound and the isocyanate-containing functionalizing compound is via the oxygen atom from an —OH group of the argentatin compound to the carbon atom in the carbonyl of the isocyanate group of the functionalizing compound. As a non-limiting example, use of a diisocyanate (in the form of TDI) for the isocyanate-containing functioning compound could result in the following modified or functionalized argentatin structure when an excess of TDI was present:

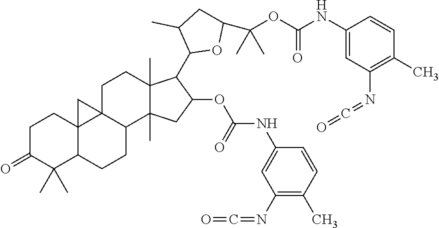 [SMILES: Cc1ccc(NC(=O)OC2CC3(C)C4CCC5C(C)(C)C(=O)CCC56CC46CCC3(C)C2C2OC(C(C)(C)OC(=O)Nc3ccc(C)c(N=C=O)c3)CC2C)cc1N=C=O]

The above structure (structure I) represents a functionalized form of Argentatin A wherein two of the —OH groups of the Argentatin A have (each) bonded to an isocyanate group of an isocyanate-containing functionalizing compound (with molecules of the isocyanate-containing functionalizing compound being shown). As another non-limiting example of the functionalization reactions that occur, use of a diisocyanate (e.g., in the form of TDI) for the isocyanate-containing functioning compound could result in the following modified or functionalized argentatin structure when an excess of Argentatin A was present:

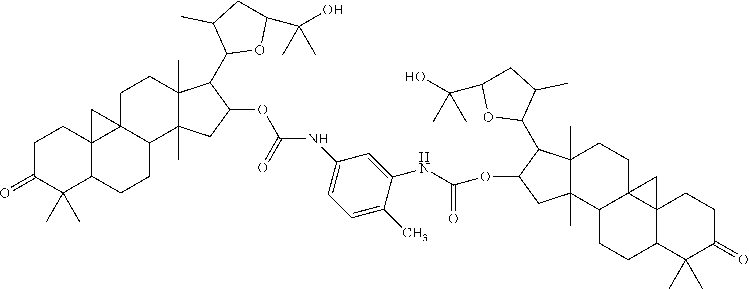 [SMILES: Cc1ccc(NC(=O)OC2CC3(C)C4CCC5C(C)(C)C(=O)CCC56CC46CCC3(C)C2C2OC(C(C)(C)O)CC2C)cc1NC(=O)OC1CC2(C)C3CCC4C(C)(C)C(=O)CCC45CC35CCC2(C)C1C1OC(C(C)(C)O)CC1C]

The above structure (Structure II) represents a functionalized form of Argentatin A wherein two Argentatin A molecules have bonded to one molecule of TDI, with the bonding occurring between the hydrogen in one —OH group of each argentatin and the carbon in both of the carbonyl groups in the isocyanate groups of TDI. It should be understood that the modified guayule resin product according to the first and second embodiments disclosed herein will generally contain additional forms of functionalized argentatins (and other resin moieties), e.g., from reaction of other argentatins (or other resin moieties) with the isocyanate-containing functionalizing compound, through the reaction of —OH groups with the isocyanate groups. These other resin moieties refers to additional components that may be present in the guayule resin and that contain one or more —OH groups (e.g., Guayulin C, Guayulin D, sesquiterpenes such as partheniol, eudesmanes such as eudesmol, and polyphenols such as tannins and flavonols). In addition, further reactions can occur between the argentatins (or other resin moieties) and isocyanate-containing functionalizing compounds to provide low molecular weight oligomers wherein repeat units of both an argentatin (or other resin moiety) and the isocyanate-containing functionalizing compound would be present. Generally, the molecular weight of such low molecular weight oligomers will be greatest when the molar amounts of a di-OH argentatin, such as Argentatin A, and a diisocyanate, such as TDI, are relatively equal (e.g., around 1/1). For argentatin compounds (or other resin moieties) which contain more than one —OH group, more than one point of attachment to the isocyanate group of the isocyanate-containing functionalizing compound is possible. For argentatin compounds (or other resin moieties) with only one —OH group, only one point of attachment to the isocyanate group of the isocyanate-containing functionalizing compound is possible and thus the degree of oligomerization possible may be limited. Overall, the bonding between the argentatins and the isocyanate group (or more broadly, the isocyanate-containing functionalizing compound) results in a modified guayule resin product. Thus, as should be apparent from the above description, by controlling the relative amounts of argentatins and isocyanate-containing functionalizing compound, it is possible to influence the structure of the modified (functionalized) argentatins and as a result influence the molecular weight of the modified guayule resin product.

In certain embodiments of the first and second embodiments, the modified guayule resin product contains a majority by weight (e.g., 51%, 55%, 60%, 65%, 70%, 75%, 80%, or more), preferably at least 60% by weight (e.g., 60%, 65%, 70%, 75%, 80%, 85% or more) of the modified guayule resin in an oligomer form (i.e., a low molecular weight oligomer) of [argentatin-FC]n wherein FC represents the isocyanate-containing functionalizing compound which is bonded to an argentatin and n is an integer of 2 to 50 (e.g., 2, 4, 6, 8, 10, 12, 14, 16, 18, 20, 22, 24, 25, 26, 28, 30, 32, 34, 36, 38, 40, 42, 44, 46, 48, or 50), preferably an integer of 2 to 25 (e.g., 2, 3, 4, 5, 6, 7, 8, 9, 10, 11, 12, 13, 14, 15, 16, 17, 18, 19, 20, 21, 22, 23, 24, or 25). The foregoing percentages by weight are based upon the total weight of the modified guayule resin product.

According to the first and second embodiments, the particular isocyanate-containing functionalizing compound used to functionalize the argentatins may vary. In certain embodiments of the first and second embodiments disclosed herein, the isocyanate-containing functionalizing compound is a mono-isocyanate, a diisocyanate, a triisocyanate, or a combination thereof, each as discussed in more detail below. In preferred embodiments of the first and second embodiments, the isocynate-containing functionalizing compound is preferably a diisocyanate, a triisocyanate, or a combination thereof.

According to the first and second embodiments, the percentage by mass or weight of the hydrogen atoms from the —OH groups of the argentatins that are replaced by a functional group from the isocyanate-containing functionalizing compound may vary. In preferred embodiments of the first and second embodiments, the mixture of functionalized argentatins in the modified guayule resin product have at least 50% (e.g., 50%, 55%, 60%, 65%, 70%, 75%, 80%, 85%, 90%, or more) of their hydrogen atoms from their —OH groups replaced with the at least one functional group, preferably 60 to 90% (e.g., 60, 65, 70, 75, 80, 85, or 90%) of their hydrogen atoms from their —OH groups replaced with the at least one functional group. As a non-limiting example, if the total quantity of —OH groups in a sample containing a mixture of —OH group containing argentatins were 100 —OH groups, then having 55% of the hydrogen atoms from the —OH groups replaced with an isocyanate group would mean that 55 of the 100 —OH groups had a hydrogen atom replaced with an isocyanate group. The percentage of hydrogen atoms from the —OH groups of the mixture of argentatins that is replaced by at least one functional group from an alkoxysilane can be considered to be the percentage of functionalization of the mixture of argentatins. In other words, a mixture of argentatins wherein 75% of hydrogen atoms from their —OH groups have been replaced with a functional group from the functionalizing can be considered to be 75% functionalized.

According to the first and second embodiments disclosed herein, the properties (e.g., Tg, Mn and Mw) of the modified guayule resin product may vary. In certain preferred embodiments of the first and second embodiments, the modified guayule resin product has a Tg of 1 to 100° C. (e.g., 1, 10, 20, 30, 40, 50, 60, 70, 80, 90 or 100° C. In more preferred embodiments of the first and second embodiments, the modified guayule resin product has a Tg of 30 to 90° C. (e.g., 30, 35, 40, 45, 50, 55, 60, 65, 70, 75, 80, 85, or 90° C.), even more preferably 40 to 80°° C. (e.g., 40, 45, 50, 55, 60, 65, 70, 75, or 80° C.). In certain embodiments of the first and second embodiments, the modified guayule resin product has a Mn of 1,000 to 10,000 grams/mole (e.g., 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10000 grams/mole), preferably 1,000 to 5,000 grams/mole (e.g., 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 grams/mole). In certain embodiments of the first and second embodiments, the modified guayule resin product has a Mw of 2,000 to 15,000 grams/mole (e.g., 2000, 2500, 3000, 3500, 4000, 4500, 5000, 5500, 6000, 6500, 7000, 7500, 8000, 8500, 9000, 9500, 10000, 10500, 11000, 11500, 12000, 12500, 13000, 13500, 14000, 14500, or 15000 grams/mole). In certain embodiments of the first and second embodiments, the modified guayule resin has Mn and Mw within the foregoing ranges; in certain such embodiments, the modified guayule resin also has a Tg within one of the foregoing ranges. The Mw and Mn values can be determined by GPC analysis, e.g., using the method described in the working Examples (i.e., measured against a polystyrene standard). The Tg values can be determined by DSC, using an instrument such that as manufactured by TA Instruments (New Castle, Delaware), where the measurement is conducted using a temperature elevation of 10° C./minute after cooling at −120° C. Thereafter, a tangent is drawn to the base lines before and after the jump of the DSC curve. The temperature on the DSC curve (read at the point corresponding to the middle of the two contact points) can be used as Tg.

According to the first and second embodiments, the amount of guayule rubber that is present in the modified guayule resin product may vary. As those of skill in the art will understanding, the amount of guayule rubber that is present in the modified guayule resin product will often be dependent upon the amount of guayule rubber that was present in the (unmodified) guayule resin component from which the modified guayule resin product is prepared. However, the amount of guayule rubber present in the modified guayule resin product can also refer to guayule rubber that is added after functionalization of the guayule resin component. In certain embodiments of the first and second embodiments, the modified guayule resin product contains less than 10% by weight (e.g., 9.5%, 9%, 8.5%, 8%, 7.5%, 7%, 6.5%, 6%, 5.5%, 5%, 4.5%, 4%, 3.5%, 3%, 2.5%, 2%, 1.5%, 1%, 0.5%, or less), preferably less than 5% by weight (e.g., 4.5%, 4%, 3.5%, 3%, 2.5%, 2%, 1.5%, 1%, 0.5%, or less) or even less than 1% by weight of guayule rubber.

According to the first and second embodiments disclosed herein, the percentage by weight of functionalized argentatins in the modified guayule resin product may vary. Generally, the percentage by weight of functionalized argentatins (or mixture of argentatins having at least one isocyanate group) in the modified guayule resin product will be influenced by two factors, the percentage by weight of non-functionalized argentatins having —OH groups that was present in the (unmodified) guayule resin component and the amount of isocyanate-containing functionalizing compound, with the first factor having a larger influence assuming a sufficient amount of isocyanate-containing functionalizing compound is provided for reaction. Preferably, according to the first and second embodiments, the mixture of functionalized argentatins constitutes a primary component by weight of the modified guayule resin product. By primary component is meant that of the different components present in the modified guayule resin product (e.g., unmodified argentatins, triglycerides, fatty acids), the component that is present in the largest amount is the functionalized argentatin component (which will generally be a mixture of functionalized argentatins). In certain preferred embodiments of the first and second embodiments, the mixture of functionalized argentatins (or mixture of argentatins having at least one isocyanate group) constitutes at least 40% by weight of the modified guayule resin product (e.g., 40%, 50%, 60%, 70%, 80%, 90%, 95% or more), including 40-65% by weight, 40-60% by weight, 45-65% by weight and 45-60% by weight of the modified guayule resin product. In other preferred embodiments of the first and second embodiments, the mixture of functionalized argentatins (or mixture of argentatins having at least one isocyanate functional group) constitutes (includes) at least a majority by weight of the modified guayule resin product (e.g., 51%, 55%, 60%, 65%, 70%, 75%, 80%, 85%, 90%, 95% or more), preferably at least 60% by weight of the modified guayule resin product (e.g., 60%, 65%, 70%, 75%, 80%, 85%, 90%, 95% or more), at least 70% by weight (e.g., 70%, 75%, 80%, 85%, 90%, 95%, or more), including 60-95% by weight, 60-90% by weight, 60-85% by weight, 65-95% by weight, 65-90% by weight, 65-85% by weight, 70-95% by weight, 70-90% by weight, and 70-85% by weight of the modified guayule resin product. The amount of functionalized argentatins in the modified guayule resin product can be determined by GPC analysis (e.g., using the procedure described in the working Examples, including a polystyrene standard) where the functionalized argentatins will generally be represented by at least the first eluting peak and in certain instances at least a portion of the second eluting peak (the functionalized argentatins will generally have a Mn in the range of 1,000 to 10,000 grams/mole, as discussed infra, and, thus, one of skill in the art will understand how to interpret the GPC results to correspond to the amount of product within this Mn range.) For analysis of the modified guayule resin product by GPC (such as is discussed in the working Examples), the increase in the area of the first GPC peak can generally be understood as not being attributable to guayule rubber but instead to functionalized argentatins. The modified guayule resin product may also contain some portion of triglycerides, fatty acids, and unmodified argentatins and guayulins (with the relative amounts varying generally depending upon the amount present in the unmodified guayule resin component and/or any purification processes conducted on the modified guayule resin product to remove such materials.)

### Guayule Resin Component

As discussed above, the process of the second embodiment includes use of a guayule resin component which is mixed with a functionalizing compound to produce the modified guayule resin product. Similarly, the modified guayule resin product of the first embodiment can be understood as being a functionalized version of a guayule resin component wherein the functionalizing compound discussed herein is used. As mentioned above, the guayule resin component comprises (includes) a mixture of argentatins which have —OH groups. Preferably, according to the first and second embodiments, the mixture of argentatins constitutes a primary component by weight of the guayule resin component. By primary component is meant that of the different components present in the guayule resin component (e.g., argentatins, triglycerides, fatty acids, and low molecular weight rubber), the component that is present in the largest amount is the argentatin component (which will generally be a mixture of argentatins). In certain more preferred embodiments of the first and second embodiments, the mixture of argentatins having at least one —OH group constitutes at least 40% by weight of the guayule resin component (e.g., 40%, 50%, 60%, 70%, 80%, 90%, 95% or more), including 40-65% by weight, 40-60% by weight, 45-65% by weight and 45-60% by weight of the guayule resin component. In other preferred embodiments of the first and second embodiments, the mixture of argentatins having at least one —OH group constitutes a majority by weight of the guayule resin component (e.g., 51%, 55%, 60%, 65%, 70%, 75%, 80%, 85%, 90%, 95% or more) preferably at least 60% by weight of the guayule resin component (e.g., 60%, 65%, 70%, 75%, 80%, 85%, 90%, 95% or more), at least 70% by weight (e.g., 70%, 75%, 80%, 85%, 90%, 95%, or more), including 60-95%, 60-90%, 60-85%, 65-95%, 65-90%, 65-85%, 70-95%, 70-90%, and 70-85% by weight of the guayule resin component. Generally, due to the difficulty in separating the argentatin component of guayule resin from other naturally occurring components, the percentage by weight of the guayule resin component which constitutes argentatins with —OH groups will be less than 100%. Other resin moieties that may be present in the guayule resin component include, but are not limited, to other —OH group containing moieties such as Guayulin C, Guayulin D, sesquiterpenes such as partheniol, eudesmanes such as eudesmol, and polyphenols such as tannins and flavonols; generally, the total amount of such other —OH group containing moieties in the guayule resin component will be less than the total percentage by weight of —OH group containing argentatins. The guayule resin component may also contain other non-reactive moieties such as Guayulin A, Guayulin B, triglycerides, and non —OH group containing terpene compounds; generally the total amount of such non-reactive guayule moieties will be less than the total percentage by weight of —OH group containing argentatins.

According to the first and second embodiments, the amount of guayule rubber that is present in the guayule resin component may vary. In certain embodiments of the first and second embodiments, the guayule resin component contains less than 10% by weight (e.g., 9.5%, 9%, 8.5%, 8%, 7.5%, 7%, 6.5%, 6%, 5.5%, 5%, 4.5%, 4%, 3.5%, 3%, 2.5%, 2%, 1.5%, 1%, 0.5%, or less), preferably less than 5% by weight (e.g., 4.5%, 4%, 3.5%, 3%, 2.5%, 2%, 1.5%, 1%, 0.5%, or less) or even less than 1% by weight of guayule rubber.

### Functionalizing Compound

As discussed above, according to the first and second embodiments, the functionalizing compound is an isocyanate-containing compound. According to the first and second embodiments, the particular isocyanate-containing functionalizing compound used to functionalize the argentatins may vary. In certain embodiments of the first and second embodiments disclosed herein, the isocyanate-containing functionalizing compound is a mono-isocyanate, a diisocyanate, a triisocyanate, or a combination thereof, each as discussed in more detail below. In preferred embodiments of the first and second embodiments, the isocyanate-containing functionalizing compound is preferably a diisocyanate, a triisocyanate, or a combination thereof.

In certain embodiments of the first and second embodiment, the isocyanate-containing functionalizing compound is aliphatic or cycloaliphatic. In other words, an aliphatic isocyanate-containing functionalizing compound can be understood as one that has exclusively (only) isocyanate groups that are bound to straight or branched carbon chains and a cycloaliphatic isocyanate-containing compound can be understood as one that has an isocyanate group bound to at least one cycloaliphatic ring system. In preferred embodiments of the first and second embodiments when the isocyanate-containing functionalizing compound is aliphatic or cycloaliphatic, the isocyanate-containing functionalizing compound is an aromatic compound containing at least one isocyanate functional group and an aromatic group with 4-20 carbon atoms (e.g., 4, 5, 6, 7, 8, 9, 10, 11, 12, 13, 14, 15, 16, 17, 18, 19, or 20 carbon atoms). In more preferred embodiments of the first and second embodiments when the isocyanate-containing functionalizing compound is aliphatic or cycloaliphatic, the isocyanate-containing functionalizing compound is selected from the group consisting of: trimethylene diisocyanate (also known as 1,3-diisocyanopropane), tetramethylene diisocyanate (also known as 1,4-diisocyanobutane), pentamethylene diisocyanate (also known as 1,5-diisocyanatopentane), 2-methyl-1,5-diisocyanatopentane, hexamethylene diisocyanate (also known as 1,6-diisocyanatohexane), trimethylhexamethylene diisocyanates (e.g., 2,2,4-trimethylhexamethylene diisocyanate and 2,4,4-trimethylhexamethylene diisocyanate) octamethylene 1,8-diisocyanate, nonamethylene 1,9-diisocyanate (also known as 1,9-diisocyanononane), decamethylene 1,10-diisocyanate (also known as 1,10-diisocyanatodecane), dodecamethylene 1,12-diisocyanate (also known as 1,12-diisocyanatododecane), tetradecamethylene 1,14=diisocyanate (also known as 1,14-diisocyanatotetradecane), cyclohexane diisocyanates (e.g., 1,4-cyclohexane diisocyanate, 1,3-cyclohexane diisocyanate, 1,2-cyclohexane diisocyanate, 1-isocyanato-3,3,5-trimethyl-5-(isocyanatomethyl)cyclohexane, 1,3-bis(isocyanatomethyl)cyclohexane, 1,4-bis(isocyanatomethyl)cyclohexane, 2,4-diisocyanato-1-methylcyclohexane, or 2,6-diisocyanato-1-methylcyclohexane), dicyclohexane methane diisocyanates (e.g., 4,4′-di(isocyanatocyclohexyl)-methane and or 2,4′-di(isocyanatocyclohexyl)-methane), dicyclohexylmethane diisocyanates (e.g., 4,4′-dicyclohexylmethane diisocyanate), isophorone diisocyanate (also known as IPDI), tetramethylxylylene diisocyanate (also known as TMXDI), trimethylhexane diisocyanate, tetramethylhexane diisocyanate, 2,4,6-trioxotriazine-1,3,5(2H,4H,6H)-triyl)tris(hexamethylene)isocyanate, 1,3,5-tris(6-isocyantohexyl)-1,3,5-triazinane-2,4,6-trione (also known as HDI isocyanurate), and combinations thereof.

In certain embodiments of the first and second embodiment, the isocyanate-containing functionalizing compound is aromatic. In other words, an aromatic isocyanate-containing functionalizing compound can be understood as one which has at least one isocyanate group bound to at least one aromatic ring system. In preferred embodiments of the first and second embodiments when the isocyanate-containing functionalizing compound is aromatic, the isocyanate-containing functionalizing compound is an aromatic compound containing at least one isocyanate functional group and an aromatic group with 6-20 carbon atoms (e.g., 6, 7, 8, 9, 10, 11, 12, 13, 14, 15, 16, 17, 18, 19, or 20 carbon atoms). In more preferred embodiments of the first and second embodiments when the isocyanate-containing functionalizing compound is aromatic, the isocyanate-containing functionalizing compound is selected from the group consisting of: p-phenylene diisocyanate, toluene diisocyanate (also known as TDI with common forms being 2,4-toluene diisocyanate and 2,6-toluene diisocyanate), xylylene diisocyanate, naphthyl isocyanate (e.g., 1-naphthyl isocyanate and 2-naphthyl isocyanate), naphthyl diisocyanate (also known as NDI, with a common form being 1,5-naphthalene diisocyanate), diphenylmethane diisocyanate (also known as MDI, with common forms being 2,4′-diphenylmethane diisocyanate and 4,4′-diphenylmethane diisocyanate), 3,3′-methyleneditoluene-4,4′-diisocyanate, toluenediisocyanate-trimethylolpropane adduct, 4,4′-diphenylether diisocyanate, tetrachlorophenylene diisocyanate, 3,3′-dichloro-4,4′-diphenylmethane diisocyanate, triphenylmethane triisocyanate, triisocyanate phenylthiophosphate, tris(4-isocyanatophenyl)thiophosphate, 1,3,5-tris(6-isocyanatohexyl)biuret (HDI biuret), and combinations thereof.

In certain embodiments of the first and second embodiments disclosed herein, the isocyanate-containing functionalizing compound is a diisocyanate. In certain embodiments of the first and second embodiments, it may be preferred to use a diisocyanate as the isocyanate-containing functionalizing compound due to the commercial availability of such compounds. According to the first and second embodiments, one or more than one diisocyanate may be used as the isocyanate-containing functionalizing compound. In addition, in certain embodiments of the first and second embodiments, the isocyanate-containing functionalizing compound is a combination of at least one diisocyanate compound and at least one triisocyanate compound; in certain such embodiments, the at least one diisocyanate compound is present in a majority amount (by weight) based upon the total amount of isocyanate-containing functionalizing compound. Exemplary diisocyanate compounds suitable for use as the isocyanate-containing functionalizing compound include, but are not limited to, trimethylene diisocyanate (also known as 1,3-diisocyanopropane), tetramethylene diisocyanate (also known as 1,4-diisocyanobutane), pentamethylene diisocyanate (also known as 1,5-diisocyanatopentane), 2-methyl-1,5-diisocyanatopentane, hexamethylene diisocyanate (also known as 1,6-diisocyanatohexane or HDI), trimethylhexamethylene diisocyanates (e.g., 2,2,4-trimethylhexamethylene diisocyanate and 2,4,4-trimethylhexamethylene diisocyanate) octamethylene 1,8-diisocyanate, nonamethylene (also known as 1,9-diisocyanotononane), decamethylene (also known as 1,10-diisocyanate decane), dodecamethylene (also known as 1,12-diisocyanate dodecane), tetradecamethylene (also known as 1,14-diisocyanate tetradecane), cyclohexane diisocyanates (e.g., 1,4-cyclohexane diisocyanate, 1,3-cyclohexane disocyanate, 1,2-cyclohexane diisocyanate, 1-isocyanato-3,3,5-trimethyl-5-(isocyanatomethyl)cyclohexane, 1,3-bis(isocyanatomethyl)cyclohexane, 1,4-bis(isocyanatomethyl)cyclohexane, 2,4-diisocyanato-1-methylcyclohexane, or 2,6-diisocyanato-1-methylcyclohexane), dicyclohexane methane diisocyanates (e.g., 4,4′-di(isocyanatocyclohexyl)-methane (also known as HMDI) and or 2,4′-di(isocyanatocyclohexyl)-methane), dicyclohexylmethane diisocyanates (e.g., 4,4′-dicyclohexylmethane diisocyanate), isophorone diisocyanate (also known as IPDI), tetramethylxylylene diisocyanate (also known as TMXDI), trimethylhexane diisocyanate, tetramethylhexane diisocyanate, p-phenylene diisocyanate, toluene diisocyanate (also known as TDI with common forms being 2,4-toluene diisocyanate and 2,6-toluene diisocyanate), xylylene diisocyanate, naphthyl isocyanate (e.g., 1-naphthyl isocyanate and 2-naphthyl isocyanate), naphthyl diisocyanate (also known as NDI, with a common form being 1,5-naphthalene diisocyanate), diphenylmethane diisocyanate (also known as MDI, with common forms being 2,4′-diphenylmethane diisocyanate and 4,4′-diphenylmethane diisocyanate), 3,3′-methyleneditoluene-4,4′-diisocyanate, toluenediisocyanate-trimethylolpropane adduct, 4,4′-diphenylether diisocyanate, lysine diisocyanate, tetrachlorophenylene diisocyanate, 3,3′-dichloro-4,4′-diphenylmethane diisocyanate, and combinations thereof. In preferred embodiments of the first and second embodiments when the isocyanate-containing functionalizing compound is a diioscyanate, it is selected from the group consisting of hexamethylene diisocyanate (also known as 1,6-diisocyanatohexane or HDI), dicyclohexane methane diisocyanates (e.g., 4,4′-di(isocyanatocyclohexyl)-methane or HMDI, and 2,4′-di(isocyanatocyclohexyl)-methane), isophorone diisocyanate (also known as IPDI), toluene diisocyanate (also known as TDI with common forms being 2,4-toluene diisocyanate and 2,6-toluene diisocyanate), naphthyl diisocyanate (also known as NDI, with a common form being 1,5-naphthalene diisocyanate), diphenylmethane diisocyanate (also known as MDI, with common forms being 2,4′-diphenylmethane diisocyanate and 4,4′-diphenylmethane diisocyanate), and combinations thereof.

In certain embodiments of the first and second embodiments disclosed herein, the isocyanate-containing functionalizing compound is a triisocyanate. In certain embodiments of the first and second embodiments, it may be preferred to use a triisocyanate as the isocyanate-containing functionalizing compound since such compounds may provide a benefit through the ability of their three isocyanate group to react with a portion of guayule impurities or mono —OH guayule resin moieties without causing termination of oligomerization or polymerization (which would occur with a mono-isocyanate compound and be more likely to occur with a diisocyanate compound than the triisocyanate compound). According to the first and second embodiments, one or more than one triisocyanate may be used as the isocyanate-containing functionalizing compound. In addition, in certain embodiments of the first and second embodiments, the isocyanate-containing functionalizing compound is a combination of at least one triisocyanate compound and at least one diiisocyanate compound in certain such embodiments, the at least one diisocyanate compound is present in a majority amount (by weight) based upon the total amount of isocyanate-containing functionalizing compound. In preferred embodiments of the first and second embodiments when the isocyanate-containing functionalizing compound is a diioscyanate, it is selected from the group consisting of triphenylmethane triisocyanate, lysine triisocyanate, triisocyanate phenylthiophosphate, and combinations thereof.

### Processes for Preparing a Modified Guayule Resin Product

As discussed above, the second embodiment disclosed herein is directed to a process for preparing a modified guayule resin product. Generally, according to the process of the second embodiment, a guayule resin component is provided which comprises a mixture of argentatins having —OH groups, the guayule resin component can be considered to be a starting product. The —OH groups of the argentatins should be understood as an inherent part of the argentatin structure and provide a site for bonding to the isocyanate-containing functionalizing compound, as discussed further infra. According to the process of the second embodiment, the guayule resin component is mixed with an isocyanate-containing functionalizing compound (as discussed in detail above), to produce a modified guayule resin product comprising functionalized argentatins with at least one functional group provided by the isocynate-containing functionalizing compound.

The amount of isocyanate-containing functionalizing compound that is used to prepare the modified guayule resin product (or more specifically the functionalized argentatins therein) may vary according to the process of the second embodiment. Generally, the amount of functionalizing compound can be calculated as a molar equivalent ratio of (total) isocyanates groups from the functionalizing compound to (total) —OH groups from the —OH group containing argentatins in the mixture of argentatins. Thus, use of an isocyanate-containing compound having relatively more isocyanate groups (e.g., 2 or 3) can require the use of fewer moles of that isocyanate-containing compound as compared to a mono-isocyanate compound (which has only one isocyanate group) to produce the equivalent functionalization percentage of a given quantity of —OH group containing argentatins. In certain embodiments of the second embodiment, the functionalizing compound is used in an amount sufficient to provide a molar ratio of isocyanate groups to —OH groups from the —OH group containing argentatins in a ratio of 1/4 to 4/1 (e.g., 1/4, 1/3.5, 1/3, 1/2.5, 1/2, 1/1.5, 1/1, 1.5/1, 2/1, 2.5/1, 3/1, 3.5/1, or 4/1), more preferably 1/3 to 3/1, and even more preferably 1/2x to 2/1. In certain preferred embodiments of the second embodiment (e.g., when the isocyanate-containing functionalizing compound is a diisocyanate), the functionalizing compound is used in an amount sufficient to provide a molar equivalent ratio of diisocyanate groups from the isocyanate-containing functionalizing compound to di —OH groups from the Argentatin A portion of the guayule resin component (since each diisocyanate molecule contains two isocyanate groups and each Argentatin A molecule contains two —OH groups, this ratio can also be understood as a molar ratio of diisocyanate to Argentatin A) in the mixture of argentatins of 1/6 to 6/1 (e.g., 1/6, 1/5.5, 1/5, 1/4.5, 1/4, 1/3.5, 1/3, 1/2.5, 1/2, 1/1.5, 1/1, 1.5/1, 2/1, 2.5/1, 3/1, 3.5/1, 4/1, 4.5/1, 5/1, 5.5/1 or 6/1), more preferably of 1/3 to 3/1 (e.g., 1/3, 1/2.5, 1/2, 1/1.5, 1/1, 1.5/1, 2/1, 2.5/1, or 3/1), even more preferably 1/2 to 2/1 (e.g., 1/2, 1/1.9, 1/1.8, 1/1.7, 1/1.6, 1/1.5, 1/1.4, 1/1.3, 1/1.2, 1/1.1, 1/1, 1.1/1, 1.2/1, 1.3/1, 1.4/1, 1.5/1, 1.6/1, 1.7/1, 1.8/1, 1.9/1, or 2/1). In the foregoing ratio, reference is made to the quantity (moles) of Argentatin A since in certain embodiments, a large majority either by weight or by quantity (e.g., 60% or more, 70% or more, 80% or more, etc.) of the —OH group containing argentatins in the guayule resin component will be of the Argentatin A type. As those of skill in the art would understand, in embodiment where the isocyanate-containing functionalizing compound is a triisocyanate, the use of somewhat less functionalizing compound will be needed to functionalize the same given portion of —OH group containing argentatins (or other —OH group containing resin moieties), e.g., 1/6 to 3/1 (or a ratio within that range), 1/4 to 3/1 (or a ratio within that range, or 1/2 to 2/1 (or a ratio within that range).

In certain preferred embodiments of the process of the second embodiment, heat is applied after mixing the guayule resin component with the isocyanate-containing functionalizing compound. In those embodiments of the second embodiment where heat is applied, the temperature to which the mixture (i.e., of isocyanate-containing functionalizing compound, guayule resin component, and optionally acid) is heated may vary; in preferred embodiments, the mixture is heated to a temperature of 20 to 100° C. (e.g., 20, 25, 30, 35, 40, 45, 50, 55, 60, 65, 70, 75, 80, 85, 90, 95 or 100° C.).

### Working Examples

The following examples illustrate specific and exemplary embodiments and/or features of the embodiments of the present disclosure. The examples are provided solely for the purposes of illustration and should not be construed as limitations of the present disclosure. Numerous variations over these specific examples are possible without departing from the spirit and scope of the presently disclosed embodiments. It should specifically be understood that modified guayule resin products can be prepared using different amounts of guayule resin component (containing different amounts of argentatins), as discussed above, and using different amounts or types of functionalizing compounds, also as discussed above.

Example 1 (production of a modified guayule resin product): A portion of guayule resin (a guayule resin component) was mixed with varying amounts of a functionalizing compound in the form of 2,4-toluene diisocyanate (i.e., TDI), (95% purity, obtained from Aldrich, molecular weight 174.2 grams/mole). The guayule resin component contained about 76 weight % argentatins and about 5 weight % low molecular weight rubber (determined by low molecular weight GPC). A portion of the functionalizing compound was added to a portion of the guayule resin. For example 1-A, the molar ratio of argentatin to TDI was 0.25/1 (or 1/4), for example 1-B the molar ratio of argentatin to TDI was 0.5/1 (or 1/2), and for example 1-C the molar ratio of argentatin to TDI was 0.75/1 (or 1/1.3). For ease of calculations, although the guayule resin contained a mixture of argentatins, the moles (for the molar ratio) were calculated using the molecular weight of Argentatin A (472.7 grams/mole) and assuming that all of the 76 weight % of argentatins was in the form of Argentatin A. Since the argentatins in the guayule resin were understood to be primarily a mixture of Argentatin A and Argentatin B (with molecular weights of 472.7 and 470.7 grams/mole, respectively), any percent error using this assumption was minimal. The mixture was heated in a water bath on a hot plate for about 1 hour and then placed in a vacuum oven at 110° C. to remove solvent and isolate the functionalized guayule resin product. Table 1 below shows the change in properties of the modified guayule resin product as compared to those of the guayule resin component, which change is believed to be due to the reaction of the argentatins in the guayule resin component with the functionalizing compound. As can be seen from the data in Table 1, the weight percentage of material with higher the Mn and Mw values for each of the modified guayule resin products (i.e., Examples 1-A, 1-B and 1-C) are all increased as compared to the corresponding values for the guayule resin component in unmodified form (as evidenced by the larger area percentages of the high molecular weight components 1 and 2). This increase in the weight percentage of the higher Mn and Mw components (i.e., high molecular weight (HMW) components 1 and 2) are attributed to oligomerization of the diisocyanate with the argentatins from the guayule resin. A Tg of −68.1° C. was detected in the unmodified resin component and is attributed to the presence of a small amount of guayule rubber in the resin component. Tg values of 17.4° C., 53.5° C., and 54.8° C. are observed in the modified guayule resin products which are attributed to oligomerization of the diisocyanate with the argentatins in the guayule resin. The Tg was determined by DSC and the molecular weight values were measured using GPC (with a TOSOH column and a low molecular weight (LMW) TOSOH column, THF as the eluting solvent and a polystyrene standard).

TABLE 1

| | | LMW GPC Selected Peaks | | | | | |
|---|---|---|---|---|---|---|---|
| | | HWM Component 1 | | | HMW Component 2 | | |
| | | Mn | Mw | | Mn | Mw | |
| | Tg | (grams/ | (grams/ | Area | (grams/ | (grams/ | Area |
| | ° C. | mole) | mole) | % | mole) | mole) | % |
| Guayule resin | −68.1 | 9483 | 10456 | 4.7% | 1837 | 2260 | 14.2% |
| component | | | | | | | |
| Example 1-A | 17.4 | 9343 | 10228 | 8.6% | 1894 | 2310 | 31.0% |
| Example 1-B | 53.5 | 9761 | 10702 | 21.1% | 2076 | 2561 | 39.8% |
| Example 1-C | 54.8 | 11177 | 12810 | 37.8% | 2166 | 2687 | 30.0% |

Example 2 (production of modified guayule resin products): A portion of guayule resin (a guayule resin component) was mixed with varying amounts of TDI as a functionalizing compounds. The guayule resin component used in Example 2 was different than the guayule resin component used in Example 1). For Example 2, the primary component of the guayule resin component was a mixture of argentatins, with the guayule resin containing about 50% by weight argentatins and no detectable amount of guayule rubber (determined by low molecular weight GPC). For Example 2-A, the molar ratio of argentatin to TDI was 0.9/1 (or 1/1.1), for example 2-B the molar ratio of argentatin to TDI was 1/1, and for example 2-C the molar ratio of argentatin to TDI was 1.1/1 (or 1/0.9). Mixing and heating were the same as in Example 1. Table 2 below shows the change in properties of the modified guayule resin product as compared to those of the guayule resin component, which change is believed to be due to the reaction of the argentatins in the guayule resin component with the functionalizing compound. As can be seen from the data in Table 2, the weight percentage of material with higher Mn and Mw values for each of the modified guayule resin products (i.e., Examples 2-A, 2-B and 2-C) are all increased as compared to the corresponding values for the guayule resin component in unmodified form (as evidenced by the larger area percentages of the high molecular weight components 1 and 2). This increase in the weight percentage of the higher Mn and Mw components (high molecular weight (HMW) components 1 and 2) are attributed to oligomerization of the diisocyanate with the argentatins from the guayule resin. The N/D notation for the HMW component 1 for the unmodified guayule resin is an indication that no peak was detectable. A Tg of 41.1° C. was detected in the unmodified resin component and was found to increase to Tg values of around 64° C. in the modified guayule resin products, which are attributed to oligomerization of the diisocyanate with the argentatins in the guayule resin. The Tg was determined by DSC and the molecular weight values were measured using GPC (with a TSOH column and a LMW TOSOH column, THF as eluting solvent and a polystyrene standard).

TABLE 2

| | | LMW GPC Selected Peaks | | | | | |
|---|---|---|---|---|---|---|---|
| | | HWM Component 1 | | | HWM Component 1 | | |
| | | Mn | Mw | | Mn | Mw | |
| | Tg | (grams/ | (grams/ | Area | (grams/ | (grams/ | Area |
| | ° C. | mole) | mole) | % | mole) | mole) | % |
| Guayule resin | 41.1 | N/D | N/D | N/D | 3460 | 5041 | 0.9% |
| component | | | | | | | |
| Example 2-A | 64.0 | 8280 | 8866 | 13.5% | 2571 | 2905 | 25.9% |
| Example 2-B | 64.3 | 8313 | 8897 | 14.5% | 2578 | 2912 | 26.4% |
| Example 2-C | 62 | 8269 | 8843 | 14.0% | 2575 | 2907 | 26.0% |

This application discloses several numerical range limitations that support any range within the disclosed numerical ranges, even though a precise range limitation is not stated verbatim in the specification, because the embodiments of the compositions and methods disclosed herein could be practiced throughout the disclosed numerical ranges. With respect to the use of substantially any plural or singular terms herein, those having skill in the art can translate from the plural to the singular or from the singular to the plural as is appropriate to the context or application. The various singular or plural permutations may be expressly set forth herein for sake of clarity.

It will be understood by those within the art that, in general, terms used herein, and especially in the appended claims are generally intended as “open” terms. For example, the term “including” should be interpreted as “including but not limited to,” the term “having” should be interpreted as “having at least,” the term “includes” should be interpreted as “includes but is not limited to.” It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” All references, including but not limited to patents, patent applications, and non-patent literature are hereby incorporated by reference herein in their entirety. While various aspects and embodiments of the compositions and methods have been disclosed herein, other aspects and embodiments will be apparent to those skilled in the art. The various aspects and embodiments disclosed herein are for purposes of illustration and are not intended to be limiting, with the true scope and spirit being indicated by the claims.

What is claimed is:

1-19. (canceled)

20. A modified guayule resin product comprising a mixture of argentatins having at least one functional group, wherein the at least one functional group is provided by an isocyanate-containing functionalizing compound.

21. The modified guayule resin product of claim 20, wherein the isocyanate-containing functionalizing compound is a mono-isocyanate, a diisocyanate, a triisocyanate, or a combination thereof.

22. The modified guayule resin product of claim 20, wherein the isocyanate-containing functionalizing compound is aliphatic or cycloaliphatic with 4-20 carbon atoms.

23. The modified guayule resin product of claim 20, wherein the isocyanate-containing functionalizing compound is aromatic with 6-20 carbon atoms.

24. The modified guayule resin product of claim 20, wherein the isocyanate-containing functionalizing compound is a diisocyanate.

25. The modified guayule resin product of claim 20, wherein the isocyanate-containing functionalizing compound is a triisocyanate.

26. The modified guayule resin product of claim 20, having a Tg of 1 to 100° C.

27. The modified guayule resin product of claim 20, wherein the modified guayule resin has a Mn of 1,000 to 10,000 grams/mole or a Mw of 2,000 to 15,000 grams/mole.

28. The modified guayule resin product of claim 20, wherein an oligomer form of [argentatin-FC]n wherein FC represents the isocyanate-containing functionalizing compound bonded to an argentatin and n is an integer of 2 to 50 represents a primary component by weight of the modified guayule resin product.

29. The modified guayule resin product of claim 20, wherein a majority by weight of the modified guayule resin product is present in an oligomer form of [argentatin-FC]n wherein FC represents the isocyanate-containing functionalizing compound bonded to an argentatin and n is an integer of 2 to 50, preferably 2 to 25.

30. The modified guayule resin product of claim 20 wherein the mixture of argentatins having at least one functional group have at least 50% of hydrogen atoms from their —OH groups replaced with an isocyanate group from the isocyanate-containing functionalizing compound.

31. The modified guayule resin product of claim 20, wherein less than 10% by weight of guayule rubber is present in the modified guayule resin product.

32. The modified guayule resin product of claim 20, wherein the mixture of argentatins having at least one functional group is a majority by weight of the modified guayule resin product.

33. The modified guayule resin product of claim 20, wherein the mixture of argentatins having at least one functional group is a primary component by weight.

34. A process for preparing a modified guayule resin product according to claim 20, the process comprising
a. providing a guayule resin component which comprises a mixture of argentatins having —OH groups,
b. mixing the guayule resin component with the isocyanate-containing functionalizing compound, and
c. producing the modified guayule resin product which comprises functionalized argentatins with at least one functional group provided by the isocyanate-containing functionalizing compound.

35. The process of claim 34, wherein the mixture of argentatins having —OH groups is a primary component by weight.

36. The process of claim 34, wherein the mixture of argentatins having —OH groups constitutes a majority by weight of the guayule resin component.

37. The process of claim 34, wherein the isocyanate-containing functionalizing compound is used in an amount sufficient to provide a molar equivalent ratio of isocyanate groups from the functionalizing compound to —OH groups from the mixture of argentatins of 1/4 to 4/1.

38. The process of claim 34, wherein heat is applied to a temperature of 20 to 100° C. after mixing the guayule resin component with the isocyanate-containing functionalizing compound.

39. The process of claim 34, wherein the isocyanate-containing functionalizing compound is a mono-isocyanate, a diisocyanate, a triisocyanate, or a combination thereof.
